(19) United States
(12) Patent Application Publication
(10) Pub. No.: US 2023/0059816 A1
(43) Pub. Date: Feb. 23, 2023

(54) DRY BURNING DETECTION METHOD AND APPARATUS, DRY BURNING PROTECTION METHOD AND APPARATUS, AND VAPORIZER

(71) Applicant: Shenzhen Moore Vaporization Health & Medical Technology Co., Ltd., Shenzhen (CN)

(72) Inventor: Sheng YANG, Shenzhen (CN)

(21) Appl. No.: 17/819,799

(22) Filed: Aug. 15, 2022

(30) Foreign Application Priority Data
Aug. 20, 2021 (CN) 202110960632.2

(51) Int. Cl. B05B 12/00; B05B 17/06; B05B 9/00; B05B 12/08

(52) U.S. Cl. CPC B05B 12/004; B05B 17/0607; B05B 9/002; B05B 12/08

(57) ABSTRACT

A dry burning detection method, applied to a vaporizer, includes: obtaining vaporization parameters of a vaporization piece in real time, and calculating a variance between the vaporization parameters and a sample mean, the sample mean being a value representing a vaporization parameter level of a stable vaporization stage of the vaporizer; and determining that the vaporization piece is dry-burned if the variance is greater than a preset variance value corresponding to the vaporization parameter, the preset variance value being used for distinguishing a vaporization parameter fluctuation caused by vaporization dry burning and a vaporization parameter fluctuation caused by non-vaporization dry burning.

### CROSS-REFERENCE TO PRIOR APPLICATION

Priority is claimed to Chinese Patent Application No. CN 202110960632.2, filed on Aug. 20, 2021, the entire disclosure of which is hereby incorporated by reference herein.

### FIELD

This application relates to the field of vaporization technologies, and in particular, to a dry burning detection method and apparatus and a dry burning protection method and apparatus applied to a vaporizer, a vaporizer, a computer device, and a storage medium.

### BACKGROUND

With the continuous improvement of living standards, the medical aesthetics industry is developing faster and faster, and vaporizers can help improve the aesthetics effect. For example, when a vaporizer is used to perform cold vaporization on an essence, the essence may be distributed at 100% and active ingredients in the essence may be maintained, so that the essence can be more easily absorbed by the human body, thereby exerting the maximum effect.

During use, the vaporizer can be normally vaporized only when there is the essence, and a problem of dry burning occurs when there is a lack of essence. To prevent dry burning, the existing vaporizer generally uses an electrode method to detect whether there is a lack of essence. A principle of the method is that an electrode is connected to a negative electrode of a vaporization piece through the essence, and whether there is a lack of essence in the vaporizer is determined by detecting an electrical change of the electrode.

The existing vaporizer needs to introduce the electrode to detect whether there is a lack of essence to prevent dry burning. Affected by the structure, material, and hardware circuit, etc., the implementation effect is poor.

### SUMMARY

In an embodiment, the present invention provides a dry burning detection method, applied to a vaporizer, the method comprising: obtaining vaporization parameters of a vaporization piece in real time, and calculating a variance between the vaporization parameters and a sample mean, the sample mean being a value representing a vaporization parameter level of a stable vaporization stage of the vaporizer; and determining that the vaporization piece is dry-burned if the variance is greater than a preset variance value corresponding to the vaporization parameter, the preset variance value being used for distinguishing a vaporization parameter fluctuation caused by vaporization dry burning and a vaporization parameter fluctuation caused by non-vaporization dry burning.

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 is a schematic structural diagram of a vaporizer according to an embodiment;

FIG. 2 is a schematic flowchart of a dry burning detection method according to an embodiment;

FIG. 3 is a schematic flowchart of a dry burning detection method according to another embodiment;

FIG. 4a(1) is a schematic diagram of a vaporization current generated by an influence of bubbles according to an embodiment;

FIG. 4a(2) is a schematic diagram of a signal of the vaporization current in FIG. 4a(1) after variance calculation;

FIG. 4b(1) is a schematic diagram of a vaporization current generated by being influenced by dry burning of a vaporization piece according to an embodiment;

FIG. 4b(2) is a schematic diagram of a signal of the vaporization current in FIG. 4b(1) after variance calculation;

FIG. 4c is a schematic diagram of comparison of the signals of the vaporization currents after variance calculation in FIG. 4a(2) and FIG. 4b(2);

FIG. 5 is a schematic flowchart of a dry burning detection method according to another embodiment;

FIG. 6 is a structural block diagram of a dry burning detection apparatus and a dry burning protection apparatus according to an embodiment;

FIG. 7 is a schematic flowchart of a dry burning protection method according to an embodiment;

FIG. 8 is a schematic structural diagram of a vaporizer according to another embodiment; and

FIG. 9 is a block diagram of an internal structure of a computer device according to an embodiment.

### DETAILED DESCRIPTION

In an embodiment, the present invention provides a high-precision dry burning detection method and apparatus that are simple to implement and can eliminate an influence of bubbles, a vaporizer, and a computer device.

An aspect of embodiments of this application provides a dry burning detection method, applied to a vaporizer, the method including:

obtaining vaporization parameters of a vaporization piece in real time, and calculating a variance between the vaporization parameters and a sample mean, where the sample mean is a value capable of representing a vaporization parameter level of a stable vaporization stage of the vaporizer; and

determining that the vaporization piece is dry-burned if the variance is greater than a preset variance value corresponding to the vaporization parameter, where the preset variance value is used for distinguishing a vaporization parameter fluctuation caused by vaporization dry burning and a vaporization parameter fluctuation caused by non-vaporization dry burning.

According to the dry burning detection method provided in the embodiments of this application, vaporization parameters of a vaporization piece are obtained, a corresponding variance is calculated based on the vaporization parameters, and a value of the variance is compared with a preset variance value. If the value of the variance is greater than the preset variance value, it is determined that the vaporization parameter changes greatly at this time due to dry burning of the vaporization piece, that is, the vaporization piece has a phenomenon of dry burning. The implementation process does not require additional hardware, and the implementation is fast and convenient. Moreover, in full consideration of the influence of factors such as characteristics and a structure of a liquid sol medium itself such as an essence, bubbles may be generated in a vaporization process, and the vaporization parameter may also fluctuate at the moment when the bubbles are generated. For this interference factor, in the solution provided in the embodiments of this application, a difference between a vaporization parameter fluctuation triggered by the bubbles and a vaporization parameter fluctuation triggered by dry burning is increased by using the above variance calculation manner; a value between a vaporization parameter variance corresponding to the non-vaporization dry burning condition and a vaporization parameter variance corresponding to the vaporization dry burning condition is selected as the preset variance value; and the variance corresponding to the actually obtained vaporization parameter is compared with the preset variance value during dry burning detection. In this way, dry burning determination can be quickly implemented, and the detection implementation solution well eliminates the interference of the vaporization parameter fluctuation on a detection result caused in the non-vaporization dry burning condition, thereby improving the recognition accuracy of vaporization dry burning.

In an embodiment, the step of obtaining vaporization parameters of a vaporization piece in real time, and calculating a variance between the vaporization parameters and a sample mean includes:

obtaining vaporization parameters in a first preset sampling time period, and calculating a variance between vaporization parameters at sampling moments and the sample mean; and

a second preset sampling time period includes a plurality of independent first preset sampling time periods, and the step of determining that the vaporization piece is dry-burned if the variance is greater than a preset variance value corresponding to the vaporization parameter includes:

calculating an average value of variances corresponding to vaporization parameters sampled in each of the first preset sampling time periods of the second preset sampling time period; and

determining that the vaporization piece is dry-burned if the average value of the variances is greater than the preset variance value.

In an embodiment, the vaporization parameter includes a vaporization current and/or vaporization voltage peak-to-peak value and/or vaporization power.

In an embodiment, the step of determining that the vaporization piece is dry-burned if the variance is greater than a preset variance value corresponding to the vaporization parameter includes:

obtaining an actual frequency sweep curve of the vaporization piece in a frequency sweep stage if the variance is greater than the preset variance value corresponding to the vaporization parameter, where the actual frequency sweep curve is data that reflects a change of the vaporization parameter when the vaporization piece works within a preset frequency range; and

determining that the vaporization piece is dry-burned if the actual frequency sweep curve does not match a preset frequency sweep curve, where the preset frequency sweep curve is a logic curve model of a change of a current when the vaporization piece works within the preset frequency range.

In an embodiment, the dry burning detection method further includes:

obtaining vaporization parameters of the stable vaporization stage, and determining the sample mean according to the vaporization parameters of the stable vaporization stage.

In an embodiment, the step of obtaining vaporization parameters of the stable vaporization stage, and determining the sample mean according to the vaporization parameters of the stable vaporization stage includes:

sampling a plurality of vaporization parameters in a third preset sampling time period of the stable vaporization stage; and calculating an average value of the plurality of vaporization parameters, and using the average value as the sample mean.

In an embodiment, before the step of obtaining vaporization parameters of the stable vaporization stage, the method further includes: obtaining vaporization parameters in a fourth preset sampling time period; and

determining that the vaporizer is in the stable vaporization stage if the vaporization parameters in the fourth preset sampling time period change within a preset fluctuation range. Another aspect of the embodiments of this application further provides a dry burning detection apparatus, applied to a vaporizer, the apparatus including:

a variance calculation module, configured to obtain vaporization parameters of a vaporization piece in real time, and calculate a variance between the vaporization parameters and a sample mean, where the sample mean is a value capable of representing a vaporization parameter level of a stable vaporization stage of the vaporizer; and

a dry burning determination module, configured to determine that the vaporization piece is dry-burned if the variance is greater than a preset variance value, where the preset variance value is used for distinguishing a vaporization parameter fluctuation caused by vaporization dry burning and a vaporization parameter fluctuation caused by non-vaporization dry burning.

The embodiments of this application further provide a vaporizer, including: a vaporization piece, configured to vaporize to-be-vaporized liquid;

a sampling circuit, configured to sample vaporization parameters of the vaporization piece; and

a controller, where the controller is electrically connected to the vaporization piece and the sampling circuit respectively, and is configured to drive the vaporization piece to vibrate, so that the vaporization piece vaporizes the to-be-vaporized liquid; and the controller is further configured to perform the steps of the dry burning detection method and/or the steps of the dry burning protection method.

In an embodiment, the controller in the vaporizer includes:

a drive circuit, configured to drive the vaporization piece to vibrate, so that the vaporization piece vaporizes the to-be-vaporized liquid; and

a processor, connected to the drive circuit, and configured to regulate a signal outputted by the drive circuit to the vaporization piece.

In an embodiment, the vaporization parameter includes a vaporization current and/or vaporization voltage peak-to-peak value and/or vaporization power; and the sampling circuit includes:

a first current sampling circuit, where an input end of the first current sampling circuit is electrically connected to the vaporization piece, an output end of the first current sampling circuit is connected to the processor, and the first current sampling circuit is configured to sample the vaporization current; and/or

a vaporization voltage peak-to-peak value sampling circuit, where an input end of the vaporization voltage peak-to-peak value sampling circuit is connected to an output end of the drive circuit, an output end of the vaporization voltage peak-to-peak value sampling circuit is connected to the processor, and the vaporization voltage peak-to-peak value sampling circuit is configured to sample the vaporization voltage peak-to-peak value; and/or

a voltage sampling circuit and a second current sampling circuit, where an input end of the voltage sampling circuit is connected to a vaporization power supply, an output end of the voltage sampling circuit is connected to the processor, and the voltage sampling circuit is configured to sample a vaporization input voltage; an input end of the second current sampling circuit is configured to be connected in series between the power supply and the drive circuit, and the second current sampling circuit is configured to sample a vaporization input current; and the processor is further configured to calculate the vaporization power according to the vaporization input voltage and the vaporization input current.

In an embodiment, the vaporizer further includes:

a vaporization tank, where the vaporization tank is configured to accommodate the to-be-vaporized liquid, and the vaporization piece is arranged in the vaporization tank; and

a liquid storage member, where a liquid storage cavity is formed in the liquid storage member, the liquid storage cavity is configured to store the to-be-vaporized liquid, and the liquid storage cavity is in communication with the vaporization tank.

To help understand this application, this application is described more fully with reference to the related accompanying drawings in the following. The accompanying drawings show embodiments of this application. However, this application may be implemented in many different forms, and is not limited to the embodiments described in this specification. On the contrary, the embodiments are provided to make the disclosed content of this application more thorough and comprehensive.

Unless otherwise defined, meanings of all technical and scientific terms used in this specification are the same as those usually understood by a person skilled in the art to which this application belongs. In this specification, terms used in the specification of this application are merely intended to describe objectives of the specific embodiments, but are not intended to limit this application.

It may be understood that, terms “first”, “second” and the like used in this specification may be used for describing various time periods, but these time periods are not limited by these terms. The terms are merely used for distinguishing a first time period from another time period.

It should be noted that, when an element is considered to be “connected” to another element, the element may be directly connected to the another element, or may be electrically connected to the another element through a central element. In addition, the “connection” in the following embodiments should be understood as “electrical connection”, “communication connection”, and the like if there is transmission of electrical signals or data between the connected objects.

As used herein, terms “a”, “one”, and “the” which are singular forms may also include plural forms, unless otherwise specified in the context clearly. It should further be understood that terms “comprise/include” and/or “contain” and the like indicate the presence of features, integers, steps, operations, components, parts, and/or combinations thereof, but do not preclude the possibility of the presence or addition of one or more other features, integers, steps, operations, components, parts, and/or combinations thereof. At the same time, a term “and/or” used in this specification includes any or all combinations of related listed items.

For the problems provided in the background, in an exemplary technology, an upper limit threshold and a lower limit threshold are set by obtaining an average vaporization parameter of a vaporization piece during normal vaporization. In a subsequent working process of a vaporizer, whether the vaporization piece is dry-burned is determined by monitoring whether an actual vaporization parameter of the vaporization piece exceeds the upper limit threshold and the lower limit threshold. If the vaporization parameter exceeds a range determined by the upper limit threshold and the lower limit threshold, it is determined that the vaporization piece is dry-burned.

However, the inventor found during the implementation process that, in an aesthetics vaporization project, due to the influence of factors such as characteristics, a structure of an essence itself, bubbles may be generated during a vaporization process. In this case, the vaporization parameter (such as a current, power, and a voltage peak-to-peak value of the vaporization piece) may have a relatively large increase or decrease at the moment when the bubbles are generated. When the bubbles disappear, the vaporization parameter returns to a normal working level. As a result, when the bubbles are generated, an instantaneous value of the vaporization parameter may exceed the range determined by the set upper limit threshold and the lower limit threshold, and the frequent generation of the bubbles during the vaporization process easily causes false recognition of dry burning detection, that is, a vaporization dry burning detection result has low reliability.

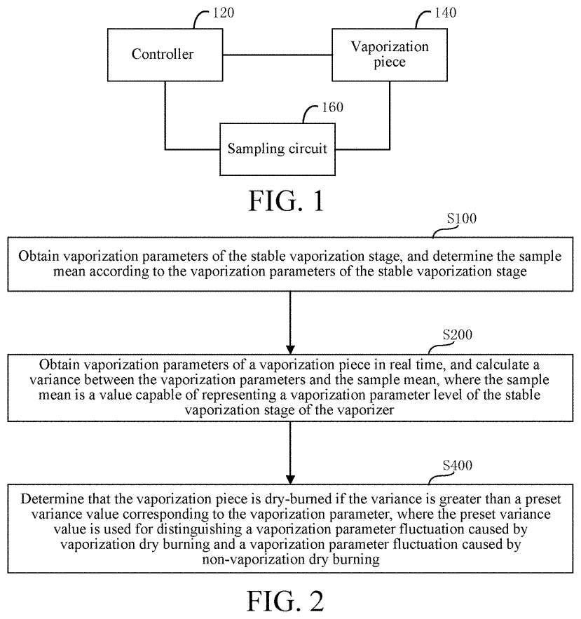

For this, in an embodiment, a dry burning detection method is provided, and the method may be applied to a vaporizer shown in FIG. 1. To implement vaporization control, the vaporizer generally includes a controller 120, a vaporization piece 140, and a sampling circuit 160. The controller 120 is configured to output a pulse width modulation (PWM) drive signal to drive the vaporization piece 140 to work. The vaporization piece 140 is configured to vaporize to-be-vaporized liquid (such as medicinal liquid, an essence, etc.) under the driving of the controller 120. The sampling circuit 160 is configured to acquire and feed back, to the controller 120, at least one of parameters such as a current flowing through the vaporization piece 140, a peak-to-peak value of a voltage of the controller 120 acting on the vaporization piece 140, and input power provided by a power supply of the vaporizer for vaporization, so that the controller 120 regulates the outputted PWM drive signal according to the vaporization parameter. It can be understood by a person skilled in the art that, FIG. 1 only shows main components for implementing vaporization control. According to functional requirements of the vaporizer, more components may be included. FIG. 1 is only for reference, to facilitate the understanding of the dry burning detection method and the dry burning protection method in the following embodiments, and does not limit the formation of the vaporizer for implementing the corresponding methods.

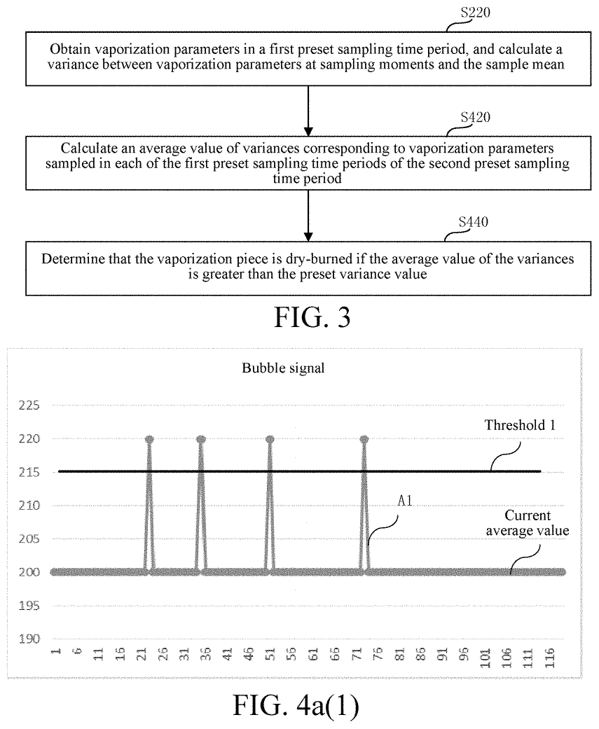

In an embodiment, as shown in FIG. 3, the dry burning detection method includes the following steps.

S200: Obtain vaporization parameters of a vaporization piece in real time, and calculate a variance between the vaporization parameters and a sample mean, where the sample mean is a value capable of representing a vaporization parameter level of a stable vaporization stage of the vaporizer.

The vaporizer is a device that can vaporize to-be-vaporized liquid. The vaporization piece may vibrate under the driving of an electric signal, and a molecular structure of the to-be-vaporized liquid in contact with the vaporization piece is broken up by using the high-frequency vibration, to generate fine mist droplets. A change of a vaporization parameter in a working process of the vaporizer compared with a vaporization parameter when the vaporizer works normally is detected, so that the determination of vaporization dry burning can be finally implemented, that is, the vaporization parameter when the vaporizer works normally provides a very important reference for dry burning detection. Based on this, by obtaining the vaporization parameter of the stable vaporization stage of the vaporizer, a value that can reflect a vaporization parameter level when the vaporizer works normally is determined as the sample mean, to provide a data basis for dry burning detection.

S400: Determine that the vaporization piece is dry-burned if the variance is greater than a preset variance value corresponding to the vaporization parameter, where the preset variance value is used for distinguishing a vaporization parameter fluctuation caused by vaporization dry burning and a vaporization parameter fluctuation caused by non-vaporization dry burning.

The variance is a variance corresponding to the vaporization parameters, namely, a mean of a sum of squares of deviations of vaporization parameters sampled in real time and the sample mean. For example, if n vaporization parameters are actually sampled during dry burning detection, a variance s² corresponding to the vaporization parameters may be calculated according to the following formula:

$${s}^{2} = \frac{{( {x}_{1} - M )}^{2} + {( {x}_{2} - M )}^{2} + {( {x}_{3} - M )}^{2} + … + {( {x}_{n} - M )}^{2}}{n}$$

x₁, x₂, . . . , xₙ are vaporization parameters obtained by n^{th} sampling, where n is greater than or equal to 1. M is the sample mean. The variance may be used for describing a degree of deviation of the actual vaporization parameters from the sample mean. Based on research and testing, the inventor found that the degree of deviation of the vaporization parameters from the sample mean during dry burning is larger than that of vaporization parameters during non-vaporization dry burning. The vaporization dry burning detection is implemented by using this difference.

The vaporization parameter fluctuation caused by the vaporization dry burning is a vaporization parameter fluctuation caused when the vaporization piece is dry-burned. The vaporization parameter fluctuation caused by the non-vaporization dry burning is a vaporization parameter fluctuation caused by factors other than dry burning of the vaporization piece, for example, a change of the vaporization parameter caused at the moment when an essence generates bubbles when the vaporization piece vaporizes the essence.

Specifically, when a vaporizer works, vaporization parameters of a vaporization piece are obtained in real time, a corresponding variance is calculated based on the vaporization parameters, and a value of the variance is compared with a preset variance value. If the value of the variance is greater than the preset variance value, it is determined that the vaporization parameter changes greatly at this time due to dry burning of the vaporization piece, that is, the vaporization piece has a phenomenon of dry burning. The implementation process does not require additional hardware, and the implementation is fast and convenient. Moreover, in full consideration of the influence of factors such as characteristics and a structure of a liquid sol medium itself such as an essence, bubbles may be generated in a vaporization process, and the vaporization parameter may also fluctuate at the moment when the bubbles are generated. For this interference factor, in the solution provided in the embodiments of this application, a difference between a vaporization parameter fluctuation triggered by the bubbles and a vaporization parameter fluctuation triggered by dry burning is increased by using the above variance calculation manner; a value between a vaporization parameter variance corresponding to the non-vaporization dry burning condition and a vaporization parameter variance corresponding to the vaporization dry burning condition is selected as the preset variance value; and the variance corresponding to the actually obtained vaporization parameter is compared with the preset variance value during dry burning detection. In this way, dry burning determination can be quickly implemented, and the detection implementation solution well eliminates the interference of the vaporization parameter fluctuation on a detection result caused in the non-vaporization dry burning condition, thereby improving the recognition accuracy of vaporization dry burning.

Based on the accurate recognition result of the vaporization dry burning, a protection action such as dry burning alarming or dry burning shutdown can be reasonably performed, to avoid damage to various components in the vaporizer caused by long-term dry burning or occurrence of safety accidents. In an embodiment, the step of obtaining the preset variance value includes:

obtaining vaporization parameters when the vaporization piece is dry burning, and calculating a dry burning signal variance corresponding to the vaporization parameters when the vaporization piece is dry burning;

obtaining vaporization parameters of the vaporization piece when to-be-vaporized liquid is triggered to generate bubbles, and calculating a bubble signal variance corresponding to the vaporization parameters of the vaporization piece generated at the moment when the to-be-vaporized liquid is triggered to generate bubbles; and

selecting a value greater than the bubble signal variance and less than the dry burning signal variance as the preset variance value.

The to-be-vaporized liquid may be selected according to an actual application scene of the vaporizer, to measure a bubble signal variance when the vaporizer is actually used, thereby providing a more targeted preset variance value for the dry burning detection during subsequent actual use of the vaporizer. For example, if the vaporizer is used in the aesthetics industry, the vaporizer is often used to vaporize the essence in common scenarios, and accordingly the essence may be selected as the to-be-vaporized liquid to perform the obtaining of bubble signal variance. In this way, when the vaporizer is used to vaporize the essence in an aesthetics salon, because the selected preset variance value is exactly a value determined for the to-be-vaporized liquid, the vaporizer can quickly and accurately determine whether there is dry burning by performing the steps of the dry burning detection method. Based on the accurate detection result, a dry burning protection action such as dry burning alarming or dry burning shutdown can be more reasonably performed during dry burning.

In an embodiment, at the time of determining the preset variance value, after a plurality of times of trials, a value greater than bubble signal variances obtained from the trials and less than dry burning signal variances obtained from the trials is selected as the preset variance value, to avoid a problem that the preset variance value is unreasonably set due to contingency of a single trial.

In addition, in an embodiment, at the time of determining the preset variance value, according to to-be-vaporized liquid commonly used in the application scenario of the vaporizer, a plurality of times of trials of the above embodiment are performed for each type of the to-be-vaporized liquid, and bubble signal variances and dry burning signal variances when each type of the to-be-vaporized liquid is trialed are obtained, to determine a value that can meet a condition of being greater than the bubble signal variances obtained from the trials of various types of to-be-vaporized liquid and less than the dry burning signal variances obtained from the trials of various types of to-be-vaporized liquid in this scenario as the preset variance value. In this way, the vaporizer performing the dry burning detection method can accurately implement dry burning determination for different types of to-be-vaporized liquid when being used in a selected application scenario. For example, when the vaporizer is used in the aesthetic salon, the preset variance value is determined by testing and obtaining dry burning signal variances and bubble signal variances of different essence products in test processes, which can ensure that the vaporizer can implement accurate dry burning detection of the vaporization piece by performing the steps of the dry burning detection method when the aesthetic salon provides essences of different brands for different users.

In an embodiment, as shown in FIG. 3, the step S200 of obtaining vaporization parameters of a vaporization piece in real time, and calculating a variance between the vaporization parameters and a sample mean includes the following step.

S220: Obtain vaporization parameters in a first preset sampling time period, and calculate a variance between vaporization parameters at sampling moments and the sample mean. The setting of the first preset sampling time period may be correspondingly configured according to a specific application scenario, for example, 15 s. The vaporization parameter in the vaporization process may be continuously obtained. However, to improve the calculation efficiency, a plurality of sampling points may be set in the first preset sampling time period, and a variance calculation may be performed according to data acquired by the sampling points, to improve the calculation efficiency, thereby improving the dry burning determination efficiency.

A second preset sampling time period includes a plurality of independent first preset sampling time periods, and the step S400 of determining that the vaporization piece is dry-burned if the variance is greater than a preset variance value corresponding to the vaporization parameter includes the following steps.

S420: Calculate an average value of variances corresponding to vaporization parameters sampled in each of the first preset sampling time periods of the second preset sampling time period. The second preset sampling time period includes at least one first preset sampling time period. For example, the second preset sampling time period may include four first preset sampling time periods, the second preset sampling time period is 40 s, each first preset sampling time period is 8 s, a time interval between two adjacent first preset sampling time periods is 2 s, and in each first preset sampling time period, the sampling is performed every two seconds, for a total of four times of sampling.

S440: Determine that the vaporization piece is dry-burned if the average value of the variances is greater than the preset variance value.

According to the dry burning detection method provided in this embodiment of this application, a plurality of times of sampling are performed in the first preset sampling time period, and variances of sampled vaporization parameters in the first preset sampling time period are calculated, to thereby eliminate a problem of unreliable detection caused by contingency of single measurement. Moreover, by performing at least one time of sampling in the second preset sampling time period, for example, four times of parameter sampling in the first preset sampling time period as described above, and synthesizing the plurality of variances obtained in the second preset sampling time period, an average value of the variances is calculated, which can avoid false detection caused by occurrence of accidental events in the first preset sampling time period. Finally, comparison is performed based on the average value of the variances and a preset variance value. If the average value is greater than the preset variance value, it is determined that the vaporization piece is dry-burned. Based on this design concept, the vaporizer performing the steps of the dry burning detection method can accurately implement dry burning detection of the vaporization piece, and perform a dry burning protection action when it is determined that the vaporization piece is dry-burned. For example, the dry burning protection action may include sound and light alarming, power-off protection, and the like.

In an embodiment, the vaporization parameter includes a vaporization current, a vaporization voltage peak-to-peak value or vaporization power. The vaporization current is a current signal flowing through the vaporization piece. The vaporization voltage peak-to-peak value is a peak-to-peak value of a voltage signal inputted to the vaporization piece. The vaporization power is power corresponding to an input voltage and an input current provided for the vaporizer by a power supply that powers the vaporizer. When different vaporization parameters are selected, the preset variance value also changes accordingly. For example, when the vaporization current is selected as the vaporization parameter for dry burning detection, the preset variance value corresponding to the vaporization parameter is a preset value corresponding to the vaporization current, that is, the preset variance value is a value capable of distinguishing a vaporization current fluctuation caused by vaporization dry burning and a vaporization current fluctuation caused by non-vaporization dry burning. Correspondingly, when the preset variance value is determined by using the steps described in the above embodiments, the sampled vaporization parameter is also the vaporization current.

In an embodiment, the step of determining that the vaporization piece is dry-burned if the variance is greater than a preset variance value includes: determining that the vaporization piece is dry-burned if a variance corresponding to any type of vaporization parameter is greater than the preset variance value.

In an embodiment, the step of determining that the vaporization piece is dry-burned if the average value of the variances is greater than the preset variance value includes:

determining that the vaporization piece is dry-burned if an average value of variances corresponding to any type of vaporization parameter is greater than the preset variance value. When there are a plurality of vaporization parameters, it may be determined that the vaporization piece is dry-burned when a variance or an average value of variances corresponding to any type of vaporization parameter is greater than the preset variance value, which can ensure that the validity of dry burning detection can still be ensured when an obtaining channel of any type of vaporization parameter fails, so that the reliability of dry burning detection is improved, and the dry burning protection implemented based on the dry burning detection method is more reliable.

To better illustrate the implementation process of the dry burning detection method provided in the embodiments of this application, an example in which the vaporization parameter is the vaporization current is used for description. It should be noted that, the example herein does not limit the actual protection scope of this application. A variance between sampled vaporization currents in a time period and a sample mean (namely, a current average value) is calculated, where the current average value is a mean of currents sampled in a time period when the vaporization is in a stable state. After current mean sampling is completed, a variance between subsequently sampled currents and the current average value is calculated.

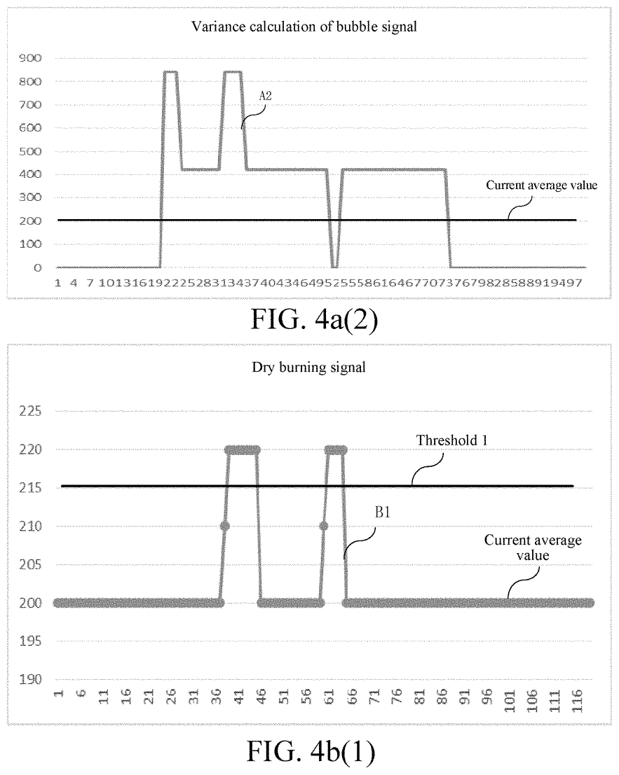

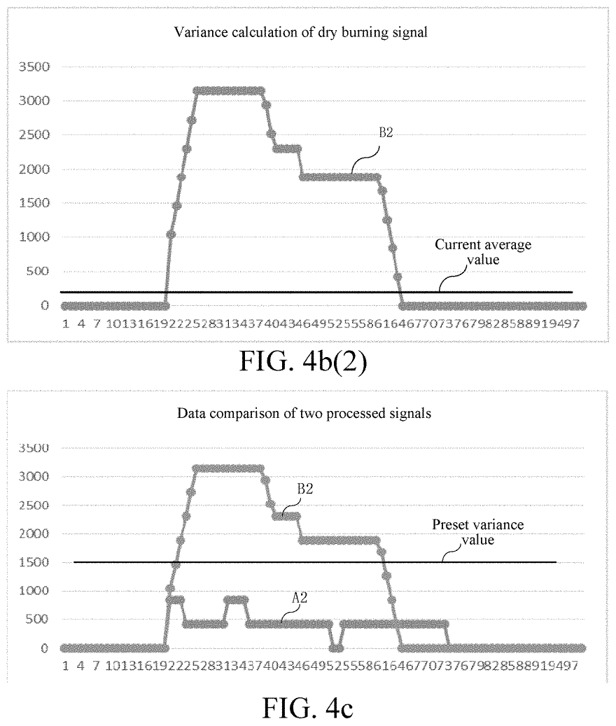

As shown in FIG. 4a(1) and FIG. 4b(1), if a threshold 1 is selected for dry burning determination, two curves A1 and B1 are respectively curves of the bubble signal and the dry burning signal, and change ranges of the two curves have little difference. If a current threshold is used for determining (that is, the threshold 1 in the figure is used for determining) whether there is vaporization dry burning, because both the two signals exceed the threshold 1, when the bubble signal causes this fluctuation, it is also determined as dry burning, resulting in false detection.

As shown in FIG. 4a(2) and FIG. 4b(2), curves A2 and B2 are correspondingly obtained after respectively calculating variances of the bubble signal A1 and the dry burning signal B1, and FIG. 4c is obtained by comparing two conditions of A2 and B2. As can be seen from a curve C1 in FIG. 4c, a difference between change ranges of vaporization current variances of A2 and B2 has a great improvement (for example, more than thrice is reached in C1) compared with that of the vaporization current curves. Through actual detected data, a value between two ranges may be set as the preset variance value, thereby accurately recognizing that the signal A1 is the bubble signal and the signal B1 is the dry burning signal, that is, it may be determined whether the vaporization parameter fluctuation is caused by vaporization dry burning, and it is determined that the vaporization piece is dry-burned when the variance is greater than the preset variance value.

An aesthetic vaporizer is used as an example. By comparison, under a condition that change ranges of currents are substantially the same, a changed vaporization current caused by dry burning is referred to as a dry burning signal herein, and it is obtained through a calculation that a range of a variance curve of the dry burning signal has a great improvement (for example, more than twice may be reached, and more than thrice is further reached in FIG. 4c) compared with a variance curve of a changed vaporization current (referred to as the a bubble signal herein) caused at the moment when essence is vaporized and generates bubbles, so that the dry burning signal and the bubble signal are distinguished more obviously, thereby improving the accuracy of system dry burning recognition.

The specific working implementation process is as follows. After vaporization of the vaporizer is stable, vaporization currents are sampled (for example, 20 pieces of data may be acquired in a third preset sampling time period), and an average value I₀ thereof is calculated as a current sample mean for variance calculation. For example, a vaporization current fluctuation in a time period may be detected, and the vaporization is considered to be stable if the fluctuation range is steady. For example, for a conventional vaporizer, a fluctuation range does not exceed ±5% when there is no bubble in normal. Therefore, when the vaporization current fluctuation does not exceed the range of ±5%, it may be determined that the vaporizer enters a stable vaporization stage, and a sample mean is calculated according to sampled vaporization currents in this stage.

In an actual use process of the vaporizer, a vaporization current Iₙk is continuously sampled, and a variance Pₙₖ of vaporization currents at sampling points is calculated. The calculation formula is as follows:

$${P}_{nk} = \frac{\sum {( {I}_{nk} - {I}_{0} )}^{2}}{n}$$

Pₙₖ represents a value of a variance obtained by sampling n vaporization parameters in a k^{th} first preset sampling time period, Iₙₖ represents a vaporization current at an n^{th} sampling point in the k^{th} first preset sampling time period, and n=1, 2, . . . , N, where N is a number of times of sampling in a single first preset sampling time period, for example, N may be equal to 20.

For a plurality of pieces of variance data (for example, k pieces of variance data) obtained in the second preset sampling time period, an average value Pᵥ thereof may be further calculated, and the calculation formula is as follows:

$${P}_{v} = \frac{\sum {p}_{nk}}{k}$$

When the average value Pᵥ of the variances exceeds a preset variance value, it is determined that the vaporization piece is in a dry-burning state.

For implementation processes of other types of vaporization parameters such as a vaporization voltage peak-to-peak value and vaporization power, reference may be made to the above explanation of the vaporization current for understanding, and details are not described herein.

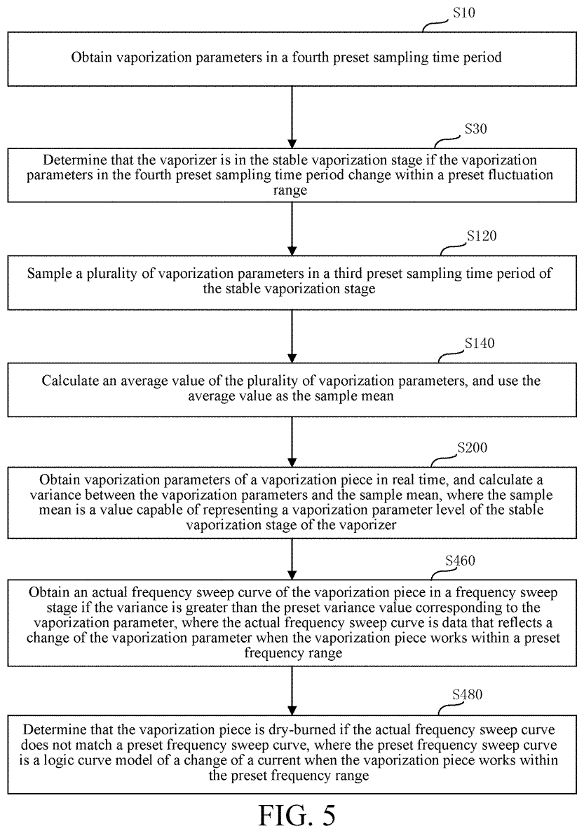

In an embodiment, as shown in FIG. 5, to further improve the accuracy and reliability of the dry burning detection result, the step S400 of determining that the vaporization piece is dry-burned if the variance is greater than a preset variance value corresponding to the vaporization parameter includes the following steps.

S460: Obtain an actual frequency sweep curve of the vaporization piece in a frequency sweep stage if the variance is greater than the preset variance value corresponding to the vaporization parameter, where the actual frequency sweep curve is data that reflects a change of the vaporization parameter when the vaporization piece works within a preset frequency range. The frequency sweep stage is a prepositive working stage for obtaining a resonant frequency of the vaporization piece. The resonant frequency of the vaporization piece is determined by controlling the vaporization piece to work at different frequencies according to the preset frequency range, so that the vaporization piece can obtain a maximum vaporization amount when working at the resonant frequency.

S480: Determine that the vaporization piece is dry-burned if the actual frequency sweep curve does not match a preset frequency sweep curve, where the preset frequency sweep curve is a logic curve model of a change of a current when the vaporization piece works within the preset frequency range. The preset frequency sweep curve is a logic curve model of a change of a current flowing through the vaporization piece when the vaporization piece works at different frequencies in the preset frequency range in a normal working state without dry burning. The logic curve model is a model for predicting a change trend of a curve, and contains determination and recognition of logic of curve features and data, and a general profile of the curve may be obtained by these features.

Because the current flowing through the vaporization piece may change in a dry-burning state and a non-dry-burning state, and in order to further improve the reliability of the dry burning detection and determination result provided in the embodiments of this application, when a variance or a mean of variances corresponding to the actual vaporization parameters is greater than the preset variance value, the actual frequency sweep curve and the preset frequency sweep curve are further compared. If the actual frequency sweep curve does not match the preset frequency sweep curve, it is determined that there is dry burning; and otherwise, it is determined that the vaporization piece is not dry-burned, and the vaporizer still remains normal working. Through the setting of the dual conditions, the accuracy of the detection result of the dry burning detection method is further improved.

In an embodiment, a preset frequency sweep curve may be established by using a starting point, an uptrend, and a downtrend. The preset frequency sweep curve is a set of a series of curves with a same profile, rather than a fixed curve.

In an embodiment, as shown in FIG. 2, the dry burning detection method further includes the following step.

S100: Obtain vaporization parameters of the stable vaporization stage, and determine the sample mean according to the vaporization parameters of the stable vaporization stage. The vaporization parameters of the stable vaporization stage are obtained and are averaged to obtain the sample mean, and then based on the variance obtained from the sample mean, a degree of deviation of actual vaporization parameters from vaporization parameters when the vaporizer normally works may be accurately reflected, thereby further ensuring the accuracy of the dry burning detection and determination result.

In an embodiment, as shown in FIG. 5, the step S100 of obtaining vaporization parameters of the stable vaporization stage, and determining the sample mean according to the vaporization parameters of the stable vaporization stage includes the following steps.

S120: Sample a plurality of vaporization parameters in a third preset sampling time period of the stable vaporization stage. The third preset sampling time period is a time period in the stable vaporization stage, and the time period may be adaptively selected by pre-configuring a number of times of sampling and a sampling frequency. For example, when the sampling is configured in a manner that a time interval between adjacent two times of sampling is 1 S and a total number of times of sampling is 15, the third preset sampling time period may be a time period from when it is determined to enter the stable vaporization stage to when a 15^{th} sampling is completed. In the third preset sampling time period, the vaporization parameters may be sampled at equal time intervals.

S140: Calculate an average value of the plurality of vaporization parameters, and use the average value as the sample mean.

According to the dry burning detection method provided in the embodiments of this application, by reasonably setting the third preset sampling time period, the reliability of the calculation result of the sample mean may be ensured, and the detection efficiency may be improved.

In an embodiment, as shown in FIG. 5, before the step S100 of obtaining vaporization parameters of the stable vaporization stage, the method further includes the following steps.

S10: Obtain vaporization parameters in a fourth preset sampling time period. Before the vaporizer is stable, the vaporization parameters thereof may fluctuate. For example, when the vaporizer is just started, in order to avoid that the sample mean calculated based on the vaporization parameters of this time period cannot accurately reflect parameter conditions of the vaporizer when working stably, the vaporization parameters in a time period may be acquired, and whether the vaporizer is in a stable working state may be determined by observing the change of the vaporization parameters.

S30: Determine that the vaporizer is in the stable vaporization stage if the vaporization parameters in the fourth preset sampling time period change within a preset fluctuation range.

The preset fluctuation range may be set correspondingly according to different specific selections of the vaporization piece. For example, in an implementation, if the vaporization current is selected as the vaporization parameter for dry burning detection, the preset fluctuation range may be set to ±5% of a stable vaporization current value. When the vaporization parameters acquired in the fourth preset sampling time period all fall within the preset fluctuation range, it is determined that the vaporizer is currently in the stable vaporization stage, and sampling can be continued to calculate the sample mean.

It is to be understood that, the steps in FIG. 2, FIG. 3, and FIG. 5 are sequentially displayed as indicated by arrows, but the steps are not necessarily sequentially performed in an order indicated by the arrows. Unless otherwise explicitly specified in this specification, execution of the steps is not strictly limited, and the steps may be performed in other sequences. In addition, at least some steps in FIG. 2 to FIG. 4, and FIG. 5 may include a plurality of steps or a plurality of stages, and these steps or stages are not necessarily performed at a same time instant, but may be performed at different time instants. The steps or stages are not necessarily performed in sequence, but may be performed by turn or alternately with other steps or at least part of steps or stages in other steps.

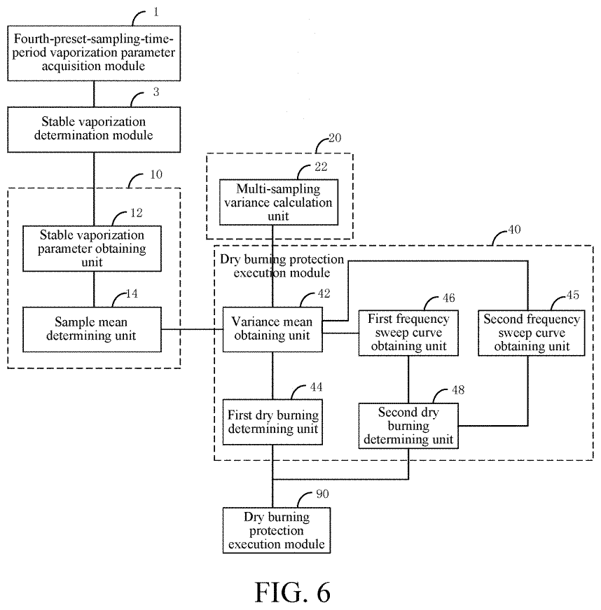

The embodiments of this application further provide a dry burning detection apparatus. As shown in FIG. 6, the dry burning detection apparatus is applied to a vaporizer, and includes:

a variance calculation module 20, configured to obtain vaporization parameters of a vaporization piece in real time, and calculate a variance between the vaporization parameters and a sample mean, where the sample mean is a value capable of representing a vaporization parameter level of a stable vaporization stage of the vaporizer; and

a dry burning determination module 40, configured to determine that the vaporization piece is dry-burned if the variance is greater than a preset variance value, where the preset variance value is used for distinguishing a vaporization parameter fluctuation caused by vaporization dry burning and a vaporization parameter fluctuation caused by non-vaporization dry burning.

In an embodiment, the variance calculation module 20 includes:

a multi-sampling variance calculation unit 22, configured to obtain vaporization parameters in a first preset sampling time period, and calculate a variance between vaporization parameters at sampling moments and the sample mean.

A second preset sampling time period includes a plurality of independent first preset sampling time periods, and the dry burning determination module 40 includes: a variance mean obtaining unit 42, configured to calculate an average value of variances corresponding to vaporization parameters sampled in each of the first preset sampling time periods of the second preset sampling time period; and

a first dry burning determining unit 44, configured to determine that the vaporization piece is dry-burned if the average value of the variances is greater than the preset variance value.

In an embodiment, when the functional units of the dry burning detection apparatus perform the steps, the used vaporization parameter includes a vaporization current and/or vaporization voltage peak-to-peak value and/or vaporization power.

In an embodiment, the dry burning determination module 40 includes:

a first frequency sweep curve obtaining unit 46, configured to obtain an actual frequency sweep curve of the vaporization piece in a frequency sweep stage if the variance is greater than the preset variance value corresponding to the vaporization parameter, where the actual frequency sweep curve is data that reflects a change of the vaporization parameter when the vaporization piece works within a preset frequency range; and

a second dry burning determining unit 48, configured to determine that the vaporization piece is dry-burned if the actual frequency sweep curve does not match a preset frequency sweep curve, where the preset frequency sweep curve is a logic curve model of a change of a current when the vaporization piece works within the preset frequency range.

In an embodiment, the dry burning determination module 40 includes:

a second frequency sweep curve obtaining unit 45, configured to obtain an actual frequency sweep curve of the vaporization piece in a frequency sweep stage if the average value of the variances is greater than the preset variance value corresponding to the vaporization parameter, where the actual frequency sweep curve is data that reflects a change of the vaporization parameter when the vaporization piece works within a preset frequency range; and

a second dry burning determining unit 48, configured to determine that the vaporization piece is dry-burned if the actual frequency sweep curve does not match a preset frequency sweep curve, where the preset frequency sweep curve is a logic curve model of a change of a current when the vaporization piece works within the preset frequency range.

In an embodiment, the dry burning detection apparatus further includes:

a sample mean obtaining module 10, configured to obtain vaporization parameters of the stable vaporization stage, and determine the sample mean according to the vaporization parameters of the stable vaporization stage.

In an embodiment, the sample mean obtaining module 10 includes:

a stable vaporization parameter obtaining unit 12, configured to sample a plurality of vaporization parameters in a third preset sampling time period of the stable vaporization stage; and

a sample mean determination unit 14, configured to calculate an average value of the plurality of vaporization parameters, and use the average value as the sample mean. In an embodiment, the dry burning detection apparatus further includes:

a fourth-preset-sampling-time-period vaporization parameter acquisition module 1, configured to obtain vaporization parameters in a fourth preset sampling time period; and a stable vaporization determination module 3, configured to determine that the vaporizer is in the stable vaporization stage if the vaporization parameters in the fourth preset sampling time period change within a preset fluctuation range.

For a specific limitation on the dry burning detection apparatus, refer to the limitation on the dry burning detection method above. Details are not described herein again. The modules in the dry burning detection apparatus may be implemented entirely or partially by software, hardware, or a combination thereof. The foregoing modules may be built in or independent of a processor of a computer device in a hardware form, or may be stored in a memory of the computer device in a software form, so that the processor invokes and performs an operation corresponding to each of the foregoing modules. It should be noted that, in this embodiment of this application, the module division is an example, and is merely logical function division, and there may be other division manners during actual application.

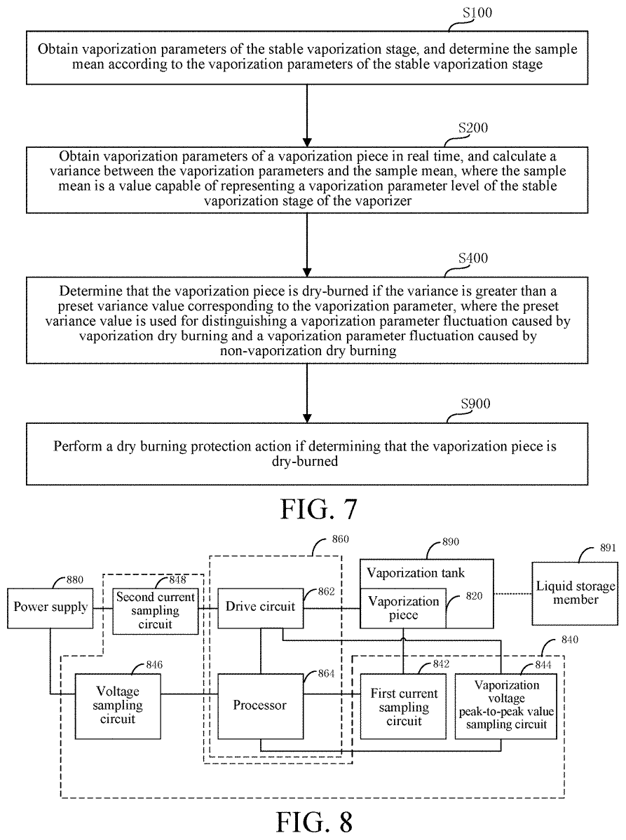

In addition, the embodiments of this application further provide a dry burning protection method. As shown in FIG. 7, the method is applied to a vaporizer, and includes the above dry burning detection method; and the dry burning protection method further includes the following step.

S900: Perform a dry burning protection action if determining that the vaporization piece is dry-burned.

The dry burning protection action is an action that can remind a user to stop dry burning of the vaporizer or can directly stop dry burning of the vaporizer. For example, the dry burning protection action may be to control a sound and light alarm set on the vaporizer to work, to remind the user to add test liquid or turn off the vaporizer; and may also be to send vaporization dry burning information to a user terminal, such as a mobile phone of the user, to remind the user to change a working state of the vaporizer. In another example, the dry burning protection action may also be to open up a communication pipeline between a liquid storage member and a vaporization tank in which the vaporization piece is located, for example, open a valve set in the pipeline, so that the test liquid stored in the liquid storage member can enter the vaporization tank in time, to change a dry-burning state of the vaporization piece, thereby protecting the vaporization piece. In another example, the dry burning protection action may also be to control the vaporizer to shut down, for example, control a switch connected in series with a channel of a power supply and a vaporizer controller to be disconnected, to stop supplying power to the vaporizer, so that the vaporization piece stops working, thereby protecting various components in the vaporizer from damage and avoiding safety accidents caused by long-term dry burning.

As for the implementation process of dry burning of the vaporization piece, reference may be made to descriptions in the above embodiments of the dry burning detection method, and details are not described herein.

The embodiments of this application further provide a dry burning protection apparatus. As shown in FIG. 6, the apparatus includes:

a variance calculation module 20, configured to obtain vaporization parameters of a vaporization piece in real time, and calculate a variance between the vaporization parameters and a sample mean, where the sample mean is a value capable of representing a vaporization parameter level of a stable vaporization stage of the vaporizer;

a dry burning determination module 40, configured to determine that the vaporization piece is dry-burned if the variance is greater than a preset variance value, where the preset variance value is used for distinguishing a vaporization parameter fluctuation caused by vaporization dry burning and a vaporization parameter fluctuation caused by non-vaporization dry burning, and

a dry burning protection execution module 90, configured to perform a dry burning protection action if it is determined that the vaporization piece is dry-burned.

For a specific limitation on the dry burning protection apparatus, refer to the limitation on the dry burning protection method and the dry burning detection method above. Details are not described herein again. The modules in the above dry burning protection apparatus may be implemented entirely or partially by software, hardware, or a combination thereof. The foregoing modules may be built in or independent of a processor of a computer device in a hardware form, or may be stored in a memory of the computer device in a software form, so that the processor invokes and performs an operation corresponding to each of the foregoing modules. It should be noted that, in this embodiment of this application, the module division is an example, and is merely logical function division, and there may be other division manners during actual application. A person skilled in the art should understand that, the dry burning protection apparatus may implement any of beneficial effects of the above dry burning detection apparatus.

In addition, the embodiments of this application further provide a vaporizer. As shown in FIG. 8, the vaporizer includes a vaporization piece 820, a sampling circuit 840, and a controller 860. The vaporization piece 820 is configured to vaporize to-be-vaporized liquid. The sampling circuit 840 is configured to sample vaporization parameters of the vaporization piece 820. The controller 860 is electrically connected to the vaporization piece 820 and the sampling circuit 840 respectively, and is configured to drive the vaporization piece 820 to vibrate, so that the vaporization piece 820 vaporizes the to-be-vaporized liquid; and the controller 860 is further configured to perform the steps of the dry burning detection method and/or the steps of the dry burning protection method.

By performing the steps in the above method embodiments and using variances to calculate a difference between an increased bubble signal and a dry burning signal, the vaporizer equipped with the above controller 860 may improve the accuracy of dry burning detection and avoid false detection of dry burning caused when bubbles are generated, thereby improving the accuracy of performing the dry burning protection action. For the specific implementation process, reference may be made to descriptions in the above method embodiments, and details are not repeated herein.

In an embodiment, as shown in FIG. 8, the controller 860 in the vaporizer may include: a drive circuit 862 and a processor 864. The drive circuit 862 is configured to drive the vaporization piece 820 to vibrate, so that the vaporization piece 820 vaporizes the to-be-vaporized liquid. The processor 864 is connected to the drive circuit 862, and is configured to regulate a signal outputted by the drive circuit 862 to the vaporization piece 820. The processor 864 can generate a drive signal to indicate the drive circuit 862 to drive the vaporization piece 820 to work at a resonant frequency, to generate a large amount of smoke and improve the use effect of the vaporizer. The drive signal may be a PWM drive signal.

In an embodiment, the vaporization parameter includes a vaporization current and/or vaporization voltage peak-to-peak value and/or vaporization power; and the sampling circuit 840 includes:

a first current sampling circuit 842, where an input end of the first current sampling circuit 842 is electrically connected to the vaporization piece 820, an output end of the first current sampling circuit 842 is connected to the processor 864, and the first current sampling circuit 842 is configured to sample the vaporization current; and/or

a vaporization voltage peak-to-peak value sampling circuit 844, where an input end of the vaporization voltage peak-to-peak value sampling circuit 844 is connected to an output end of the drive circuit 862, an output end of the vaporization voltage peak-to-peak value sampling circuit 844 is connected to the processor 864, and the vaporization voltage peak-to-peak value sampling circuit 844 is configured to sample the vaporization voltage peak-to-peak value; and/or

a voltage sampling circuit 846 and a second current sampling circuit 848, where an input end of the voltage sampling circuit 846 is connected to a vaporization power supply 880, an output end of the voltage sampling circuit 846 is connected to the processor 864, and the voltage sampling circuit 846 is configured to sample a vaporization input voltage; an input end of the second current sampling circuit 848 is configured to be connected in series between the power supply 880 and the drive circuit 862, and the second current sampling circuit 848 is configured to sample a vaporization input current; and the processor 864 is further configured to calculate the vaporization power according to the vaporization input voltage and the vaporization input current.

According to different selected vaporization parameters, different sampling circuits may be selected to sample the vaporization parameters, a variance calculation may be performed based on the vaporization parameters obtained by sampling, and dry burning detection is implemented by comparing a variance and a preset variance value. In addition, when it is determined that the vaporization piece 820 is dry-burned, a dry burning protection action may be performed. When a plurality of vaporization parameters are acquired, it may be determined that the vaporization piece 820 is dry-burned and the dry burning protection action may be performed when a variance of any type of vaporization parameter is greater than a preset variance value of the vaporization parameter. The multi-parameter detection manner can still ensure the effectiveness of the dry burning detection of the vaporizer when a certain sampling circuit fails, thereby improving the reliability of dry burning protection of the vaporizer. Various sampling circuits may feed back sampling signals thereof to the processor in the controller.

In an embodiment, the vaporizer further includes: the power supply 880, and the power supply 880 supplies power to the controller 860. The vaporizer may be configured with the power supply 880, to provide a matched working voltage for the drive circuit 862 and the processor 864 in the controller 860. The power supply 880 may include a rectification unit, a first voltage conversion unit, and a second voltage conversion unit. The rectification unit is configured to convert an external alternating current into a direct current, and transmit the direct current to the first voltage conversion unit and the second voltage conversion unit respectively. The first voltage conversion unit performs voltage conversion on the received direct current to generate a working voltage of the drive circuit 862, such as 12 V, and the second voltage conversion unit performs voltage conversion on the received direct current to generate a working voltage of the processor 864, such as 5 V, to meet requirements of the drive circuit 862 and the processor 864 for the working voltages. In an embodiment, the power supply 880 may also adopt a redundant design, to improve the working reliability of the vaporizer.

In an embodiment, the first voltage conversion unit may be an adjustable boost circuit, and the drive circuit drives the vaporization piece to work in a controller with the adjustable boost circuit and a processor.

In an embodiment, the vaporizer further includes: a vaporization tank 890 and a liquid storage member 891. The vaporization tank 890 is configured to accommodate the to-be-vaporized liquid, and the vaporization piece 820 is arranged in the vaporization tank 890. A liquid storage cavity is formed in the liquid storage member 891, the liquid storage cavity is configured to store the to-be-vaporized liquid, and the liquid storage cavity is in communication with the vaporization tank 890. Shapes of the vaporization tank 890 and the liquid storage member 891 are not limited as long as they can accommodate the to-be-vaporized liquid. Through the setting manner in which the vaporization tank 890 is in communication with the liquid storage member 891, it can be avoided that too much to-be-vaporized liquid in contact with the vaporization piece 820 influences the vaporization effect. As described in the above embodiment, when dry burning is determined through dry burning detection, a connection channel between the liquid storage member 891 and the vaporization tank 890 may be opened up, so that the to-be-vaporized liquid in the liquid storage member 891 enters the vaporization tank 890 for replenishment, thereby avoiding dry burning while ensuring the vaporization effect.

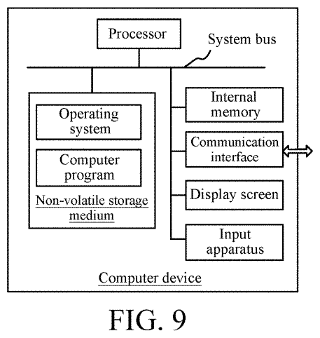

In an embodiment, a computer device is provided. The computer device may be a terminal, and an internal structure diagram thereof may be shown in FIG. 9. The computer device includes a processor, a memory, a communication interface, a display screen, and an input apparatus that are connected by using a system bus. The processor of the computer device is configured to provide computing and control capabilities. The memory of the computer device includes a non-volatile storage medium and an internal memory. The non-volatile storage medium stores an operating system and a computer program. The internal memory provides an environment for running of the operating system and the computer program in the non-volatile storage medium. The communication interface of the computer device is configured to communicate with an external terminal in a wired or wireless manner, and the wireless manner may be implemented through WIFI, a carrier network, near field communication (NFC), or other technologies. The computer program is executed by the processor to implement a dry burning detection method or a dry burning protection method. The display screen of the computer device may be a liquid crystal display screen or an electronic ink display screen. The input apparatus of the computer device may be a touch layer covering the display screen, or may be a key, a trackball, or a touch pad disposed on a housing of the computer device, or may be an external keyboard, a touch pad, a mouse, or the like. For example, a touch screen is set on a housing of the vaporizer, and the preset variance value may be configured by the touch screen, to match a dry burning detection requirement of specific types of to-be-vaporized liquid, thereby improving the accuracy of a dry burning detection result.

A person skilled in the art may understand that, the structure shown in FIG. 9 is only a block diagram of a part of a structure related to a solution of this application, and does not limit the computer device to which the solution of this application is applied. Specifically, the computer device may include more or less members than those in the drawings, or include a combination of some members, or have a different arrangement of the members.

In an embodiment, a computer device is provided, including a memory and a processor, where the memory stores a computer program, and the processor, when executing the computer program, implements the following steps.

S200: Obtain vaporization parameters of a vaporization piece in real time, and calculate a variance between the vaporization parameters and a sample mean, where the sample mean is a value capable of representing a vaporization parameter level of a stable vaporization stage of the vaporizer.

S400: Determine that the vaporization piece is dry-burned if the variance is greater than a preset variance value corresponding to the vaporization parameter, where the preset variance value is used for distinguishing a vaporization parameter fluctuation caused by vaporization dry burning and a vaporization parameter fluctuation caused by non-vaporization dry burning.

In an embodiment, the processor, when executing the computer program, further implements the following steps.

Obtain vaporization parameters when the vaporization piece is dry burning, and calculate a dry burning signal variance corresponding to the vaporization parameters when the vaporization piece is dry burning.

Obtain vaporization parameters of the vaporization piece when to-be-vaporized liquid is triggered to generate bubbles, and calculate a bubble signal variance corresponding to the vaporization parameters of the vaporization piece generated at the moment when the to-be-vaporized liquid is triggered to generate bubbles.

Select a value greater than the bubble signal variance and less than the dry burning signal variance as the preset variance value.

In an embodiment, the processor, when executing the computer program, further implements the following step.

Select, after a plurality of times of trials, a value greater than bubble signal variances obtained from the trials and less than dry burning signal variances obtained from the trials as the preset variance value.

In an embodiment, the processor, when executing the computer program, further implements the following step.

Obtain bubble signal variances and dry burning signal variances when each type of the to-be-vaporized liquid is trialed, to determine a value that can meet a condition of being greater than the bubble signal variances obtained from the trials of various types of to-be-vaporized liquid and less than the dry burning signal variances obtained from the trials of various types of to-be-vaporized liquid as the preset variance value in this scenario.

In an embodiment, the processor, when executing the computer program, further implements the following steps.

S220: Obtain vaporization parameters in a first preset sampling time period, and calculate a variance between vaporization parameters at sampling moments and the sample mean.

S420: Calculate an average value of variances corresponding to vaporization parameters sampled in each of the first preset sampling time periods of the second preset sampling time period. The second preset sampling time period includes at least one first preset sampling time period.

S440: Determine that the vaporization piece is dry-burned if the average value of the variances is greater than the preset variance value.

In an embodiment, the processor, when executing the computer program, further implements the following steps.

S460: Obtain an actual frequency sweep curve of the vaporization piece in a frequency sweep stage if the variance is greater than the preset variance value corresponding to the vaporization parameter, where the actual frequency sweep curve is data that reflects a change of the vaporization parameter when the vaporization piece works within a preset frequency range.

S480: Determine that the vaporization piece is dry-burned if the actual frequency sweep curve does not match a preset frequency sweep curve, where the preset frequency sweep curve is a logic curve model of a change of a current when the vaporization piece works within the preset frequency range.

In an embodiment, the processor, when executing the computer program, further implements the following step.

S100: Obtain vaporization parameters of the stable vaporization stage, and determine the sample mean according to the vaporization parameters of the stable vaporization stage. In an embodiment, the processor, when executing the computer program, further implements the following steps.

S120: Sample a plurality of vaporization parameters in a third preset sampling time period of the stable vaporization stage. The third preset sampling time period is a time period in the stable vaporization stage, and the time period may be adaptively selected by pre-configuring a number of times of sampling and a sampling frequency.

S140: Calculate an average value of the plurality of vaporization parameters, and use the average value as the sample mean.

In an embodiment, the processor, when executing the computer program, further implements the following steps.

S10: Obtain vaporization parameters in a fourth preset sampling time period.

S30: Determine that the vaporizer is in the stable vaporization stage if the vaporization parameters in the fourth preset sampling time period change within a preset fluctuation range.

In an embodiment, a computer-readable storage medium is provided, storing a computer program, and the computer program, when executed by a processor, implements the following steps.

S200: Obtain vaporization parameters of a vaporization piece in real time, and calculate a variance between the vaporization parameters and a sample mean, where the sample mean is a value capable of representing a vaporization parameter level of a stable vaporization stage of the vaporizer.

S400: Determine that the vaporization piece is dry-burned if the variance is greater than a preset variance value corresponding to the vaporization parameter, where the preset variance value is used for distinguishing a vaporization parameter fluctuation caused by vaporization dry burning and a vaporization parameter fluctuation caused by non-vaporization dry burning.

A person of ordinary skill in the art may understand that some or all procedures in the foregoing method embodiments may be implemented by a computer program instructing related hardware. The computer program may be stored in a non-volatile computer-readable storage medium, and when the computer program is executed, the procedures of the foregoing method embodiments may be performed. Any reference to a memory, a storage, a database, or another medium used in the embodiments provided in this application may include at least one of a non-volatile memory and a volatile memory. The non-volatile memory may include a read-only memory (ROM), a magnetic tape, a floppy disk, a flash memory, an optical memory, and the like. The volatile memory may include a random access memory (RAM) or an external cache. For the purpose of description instead of limitation, the RAM is available in a plurality of forms, such as a static RAM (SRAM) or a dynamic RAM (DRAM).

In description of this specification, description of reference terms such as “some embodiment”, “other embodiments”, or “ideal embodiments” means that specific features, structures, materials, or features described in conjunction with the embodiment or example are included in at least one embodiment or example of the present invention. In this specification, schematic descriptions of the foregoing terms do not necessarily point at a same embodiment or example.

The above methods, apparatuses and vaporizer provided in the embodiments of this application may be applied to medical, aesthetic and other fields, and in particular, to a scenario of using liquid sols such as essences that are prone to bubbles during vaporization. In the above solution provided in the embodiments of this application, high-precision dry burning detection of the above methods, apparatuses and vaporizer can be ensured, thereby avoiding frequent shutdown of the vaporizer caused by an interference of bubble signals. Based on this, the working stability of the vaporizer and user experience can be improved.

The technical features in the foregoing embodiments may be randomly combined. For concise description, not all possible combinations of the technical features in the embodiments are described. However, provided that combinations of the technical features do not conflict with each other, the combinations of the technical features are considered as falling within the scope described in this specification.

While the invention has been illustrated and described in detail in the drawings and foregoing description, such illustration and description are to be considered illustrative or exemplary and not restrictive. It will be understood that changes and modifications may be made by those of ordinary skill within the scope of the following claims. In particular, the present invention covers further embodiments with any combination of features from different embodiments described above and below. Additionally, statements made herein characterizing the invention refer to an embodiment of the invention and not necessarily all embodiments.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

What is claimed is:

1. A dry burning detection method, applied to a vaporizer, the method comprising:
obtaining vaporization parameters of a vaporization piece in real time, and calculating a variance between the vaporization parameters and a sample mean, the sample mean being a value representing a vaporization parameter level of a stable vaporization stage of the vaporizer; and
determining that the vaporization piece is dry-burned if the variance is greater than a preset variance value corresponding to the vaporization parameter, the preset variance value being used for distinguishing a vaporization parameter fluctuation caused by vaporization dry burning and a vaporization parameter fluctuation caused by non-vaporization dry burning.

2. The dry burning detection method of claim 1, wherein obtaining vaporization parameters of the vaporization piece in real time, and calculating the variance between the vaporization parameters and the sample mean comprises: obtaining vaporization parameters in a first preset sampling time period, and calculating a variance between vaporization parameters at sampling moments and the sample mean, and
wherein a second preset sampling time period comprises a plurality of independent first preset sampling time periods, and determining that the vaporization piece is dry-burned if the variance is greater than the preset variance value corresponding to the vaporization parameter comprises:
calculating an average value of variances corresponding to vaporization parameters sampled in each of the first preset sampling time periods of the second preset sampling time period; and
determining that the vaporization piece is dry-burned if an average value of the variances is greater than the preset variance value.

3. The dry burning detection method of claim 1, wherein the vaporization parameter comprises a vaporization current and/or vaporization voltage peak-to-peak value and/or vaporization power.

4. The dry burning detection method of claim 1, wherein determining that the vaporization piece is dry-burned if the variance is greater than the preset variance value corresponding to the vaporization parameter comprises:
obtaining an actual frequency sweep curve of the vaporization piece in a frequency sweep stage if the variance is greater than the preset variance value corresponding to the vaporization parameter, the actual frequency sweep curve being data that reflects a change of the vaporization parameter when the vaporization piece works within a preset frequency range; and
determining that the vaporization piece is dry-burned if the actual frequency sweep curve does not match a preset frequency sweep curve, the preset frequency sweep curve being a logic curve model of a change of a current when the vaporization piece works within the preset frequency range.

5. The dry burning detection method of claim 2, further comprising:
obtaining vaporization parameters of the stable vaporization stage, and determining the sample mean of the vaporization parameters of the stable vaporization stage.

6. The dry burning detection method of claim 5, wherein obtaining vaporization parameters of the stable vaporization stage, and determining the sample mean of the vaporization parameters of the stable vaporization stage comprises:
sampling a plurality of vaporization parameters in a third preset sampling time period of the stable vaporization stage; and
calculating an average value of the plurality of vaporization parameters, and using the average value as the sample mean.

7. The dry burning detection method of claim 2, wherein before obtaining vaporization parameters of the stable vaporization stage, the method further comprises:
obtaining vaporization parameters in a fourth preset sampling time period; and
determining that the vaporizer is in the stable vaporization stage if the vaporization parameters in the fourth preset sampling time period change within a preset fluctuation range.

8. A dry burning detection apparatus, applied to a vaporizer, the apparatus comprising:
a variance calculation module configured to obtain vaporization parameters of a vaporization piece in real time, and to calculate a variance between the vaporization parameters and a sample mean, the sample mean being a value representing a vaporization parameter level of a stable vaporization stage of the vaporizer; and
a dry burning determination module configured to determine that the vaporization piece is dry-burned if the variance is greater than a preset variance value, the preset variance value being used for distinguishing a vaporization parameter fluctuation caused by vaporization dry burning and a vaporization parameter fluctuation caused by non-vaporization dry burning.

9. A vaporizer, comprising:
a vaporization piece configured to vaporize to-be-vaporized liquid;
a sampling circuit configured to sample vaporization parameters of the vaporization piece; and
a controller electrically connected to the vaporization piece and the sampling circuit, respectively, the controller being configured to:
drive the vaporization piece to vibrate such that the vaporization piece vaporizes the to-be-vaporized liquid, and
perform the method of claim 1.

10. The vaporizer of claim 9, wherein the controller comprises:
a drive circuit configured to drive the vaporization piece to vibrate such that the vaporization piece vaporizes the to-be-vaporized liquid; and
a processor connected to the drive circuit, the processor being configured to regulate a signal outputted by the drive circuit to the vaporization piece.

11. The vaporizer of claim 10, wherein the vaporization parameter comprises a vaporization current and/or vaporization voltage peak-to-peak value and/or vaporization power, and
wherein the sampling circuit comprises:
a first current sampling circuit, an input end of the first current sampling circuit being electrically connected to the vaporization piece, an output end of the first current sampling circuit being connected to the processor, and the first current sampling circuit being configured to sample the vaporization current; and/or
a vaporization voltage peak-to-peak value sampling circuit, an input end of the vaporization voltage peak-to-peak value sampling circuit being connected to an output end of the drive circuit, an output end of the vaporization voltage peak-to-peak value sampling circuit being connected to the processor, and the vaporization voltage peak-to-peak value sampling circuit being configured to sample the vaporization voltage peak-to-peak value; and/or
a voltage sampling circuit and a second current sampling circuit, an input end of the voltage sampling circuit being connected to a vaporization power supply, an output end of the voltage sampling circuit being connected to the processor, and the voltage sampling circuit being configured to sample a vaporization input voltage; an input end of the second current sampling circuit being configured to be connected in series between the power supply and the drive circuit and the second current sampling circuit being configured to sample a vaporization input current; and the processor being further configured to calculate a vaporization power according to the vaporization input voltage and the vaporization input current.

12. The vaporizer of claim 9, further comprising:
a vaporization tank configured to accommodate the to-be-vaporized liquid, the vaporization piece being arranged in the vaporization tank; and
a liquid storage member, a liquid storage cavity being formed in the liquid storage member, the liquid storage cavity being configured to store the to-be-vaporized liquid, and the liquid storage cavity being in communication with the vaporization tank.
